(19) United States
(12) Patent Application Publication
(10) Pub. No.: US 2025/0140390 A1
(43) Pub. Date: May 1, 2025

(54) INFORMATION PROCESSING APPARATUS, MUTUAL WATCHING METHOD, RECORDING MEDIUM, AND MUTUAL WATCHING SYSTEM

(71) Applicant: NEC Corporation, Tokyo (JP)

(72) Inventors: Junichi SHIMOMURA, Tokyo (JP); Toshiko Osaka, Tokyo (JP)

(73) Assignee: NEC Corporation, Tokyo (JP)

(21) Appl. No.: 18/924,018

(22) Filed: Oct. 23, 2024

(30) Foreign Application Priority Data
Oct. 31, 2023 (JP) 2023-186738

(51) Int. Cl. G16H 40/20; G06F 3/14; G06V 40/16

(52) U.S. Cl. CPC G16H 40/20; G06F 3/14; G06V 40/168

(57) ABSTRACT

Provided are an information processing apparatus, etc. capable of supporting mutual assistance among the members of a group (optimizing the manner of mutual assistance). The information processing apparatus includes: a target detecting section for detecting, based on the state of a member of a group, a target member in need of care provided by one or more other members of the group, the state being inferred from an image of the member; and a notifying section for notifying each member of the group of the target member detected. This information processing apparatus also makes it possible to support decision-making in the mutual assistance among the members.

### CROSS REFERENCE TO RELATED APPLICATIONS

This application is based upon and claims the benefit of priority from Japanese Patent Application No. 2023-186738 filed on Oct. 31, 2023, the disclosure of which is incorporated herein in its entirety by reference.

### TECHNICAL FIELD

The present disclosure relates to an information processing apparatus, a mutual watching method, a recording medium, and a mutual watching system.

### BACKGROUND ART

By analyzing the facial image of a person, it is possible to infer various states regarding the person. For example, Patent Literature 1 below discloses a patient surveillance apparatus that uses the facial image of a medical examinee to infer the feelings of the medical examinee and calculates, on the basis of the feeling inference result and consultation information of the examinee, a stress value indicating the degree of stress the medical examinee feels.

### CITATION LIST

### Patent Literature

### [Patent Literature 1]

Japanese Patent Application Publication Tokukai No. 2020-146345

### SUMMARY OF INVENTION

### Technical Problem

In the patient surveillance apparatus disclosed in Patent Literature 1, the division of roles is fixed such that a medical examinee (patient) being on the part of a person under surveillance and the staff or the like of a medical facility is on the part of a person conducting surveillance. However, in some cases, the form of not fixing the division of roles in mutual assistance is preferable to fixing the division of roles such that someone of a group members is on the part of a watcher and someone else is on the part of a person watched over. For example, in a case where one of the members of a group becomes mentally or physically ill, it can be preferable that among the members of the group, not a predetermined member but a member who can provide care for the ill member at the moment provides care.

The present disclosure has been made in view of the above problem, and an example object thereof is to provide, for example, an information processing apparatus which supports mutual assistance among the members of a group.

### Solution to Problem

An information processing apparatus in accordance with an example aspect of the present disclosure includes at least one processor, and the at least one processor carries out: for each member of a group, calculating a value indicating a state of the member based on an image of a face of the member, the state including at least one selected from the group consisting of stress level, degree of concentration, cognitive function, feelings, degree of arousal, and degree of tension; detecting, as a target member in need of care provided by one or more other members of the group, a member with the value calculated which is equal to or greater than a predetermined threshold; and sending an instruction to change a display position of an icon of the target member to a position within a predetermined display area set on the display area, from among icons which correspond to respective members of the group and which are displayed on a display screen that the respective members of the group are allowed to browse.

A mutual watching method in accordance with an example aspect of the present disclosure includes: at least one processor calculating, for each member of a group, a value indicating a state of the member based on an image of a face of the member, the state including at least one selected from the group consisting of stress level, degree of concentration, cognitive function, feelings, degree of arousal, and degree of tension; the at least one processor detecting, as a target member in need of care provided by one or more other members of the group, a member with the value calculated which is equal to or greater than a predetermined threshold; and the at least one processor sending an instruction to change a display position of an icon of the target member to a position within a predetermined display area set on the display area, from among icons which correspond to respective members of the group and which are displayed on a display screen that the respective members of the group are allowed to browse.

A recording medium in accordance with an example aspect of the present disclosure is a computer-readable non-transitory recording medium having recorded thereon a control program for causing a computer to carry out: for each member of a group, calculating a value indicating a state of the member based on an image of a face of the member, the state including at least one selected from the group consisting of stress level, degree of concentration, cognitive function, feelings, degree of arousal, and degree of tension; detecting, as a target member in need of care provided by one or more other members of the group, a member with the value calculated which is equal to or greater than a predetermined threshold; and sending an instruction to change a display position of an icon of the target member to a position within a predetermined display area set on the display area, from among icons which correspond to respective members of the group and which are displayed on a display screen that the respective members of the group are allowed to browse.

### Advantageous Effects of Invention

An example aspect of the present disclosure provides an example advantage of making it possible to provide a technique for supporting mutual assistance among the members of a group.

### BRIEF DESCRIPTION OF DRAWINGS

FIG. 1 is a block diagram illustrating a configuration of an information processing apparatus in accordance with the present disclosure.

FIG. 2 is a flowchart illustrating a flow of a mutual watching method in accordance with the present disclosure.

FIG. 3 is a representation of an example configuration of the mutual watching system in accordance with the present disclosure.

FIG. 4 is a block diagram illustrating a configuration of another information processing apparatus in accordance with the present disclosure.

FIG. 5 is a block diagram illustrating an example configuration of a terminal in accordance with the present disclosure.

FIG. 6 is a representation of example UI screens displayed before and after notification of a target member.

FIG. 7 is a representation of example UI screens displayed during presentation of a report, presentation of data, and a video call.

FIG. 8 is a representation of an example UI screen on which business operators pertaining to care for a target member are presented.

FIG. 9 is a representation of an example UI screen on which a divergence between the result of self-evaluation of a state and the result of inference of the state is notified.

FIG. 10 is a flowchart illustrating example processes carried out by the information processing apparatus illustrated in FIG. 4.

FIG. 11 is a flowchart illustrating example processes carried out by the terminal illustrated in FIG. 5.

FIG. 12 is a block diagram illustrating a configuration of a computer which functions as the information processing apparatuses in accordance with the present disclosure.

### EXAMPLE EMBODIMENTS

The following description will discuss example embodiments of the present invention. However, the present invention is not limited to the example embodiments described below, but can be altered by a skilled person in the art within the scope of the claims. For example, any embodiment derived by appropriately combining technical means adopted in differing example embodiments described below can be within the scope of the present invention. Further, any embodiment derived by appropriately omitting one or more of the technical means adopted in differing example embodiments described below can be within the scope of the present invention. Furthermore, the advantage mentioned in each of the example embodiments described below is an example advantage expected in that example embodiment, and does not define the extension of the present invention. That is, any embodiment which does not provide the example advantages mentioned in the example embodiments described below can also be within the scope of the present invention.

### First Example Embodiment

The following description will discuss a first example embodiment, which is an example embodiment of the present invention, in detail with reference to the drawings. The present example embodiment is basic to each of the example embodiments which will be described later. It should be noted that the applicability of each of the technical means adopted in the present example embodiment is not limited to the present example embodiment. That is, each technical means adopted in the present example embodiment can be adopted in another example embodiment included in the present disclosure, to the extent of constituting no specific technical obstacle. Further, each technical means illustrated in the drawings referred to for the description of the present example embodiment can be adopted in another example embodiment included in the present disclosure, to the extent of constituting no specific technical obstacle.

### [Configuration of Information Processing Apparatus 1]

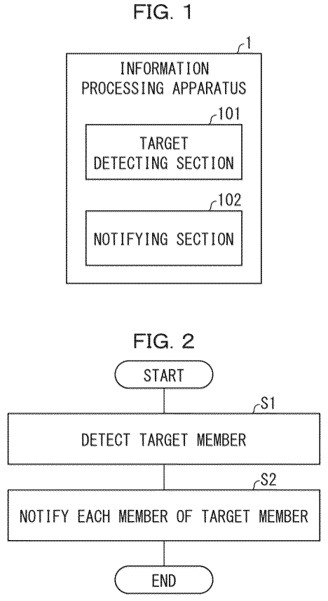

The configuration of an information processing apparatus 1 will be described below with reference to FIG. 1. FIG. 1 is a block diagram illustrating the configuration of the information processing apparatus 1. The information processing apparatus 1 includes a target detecting section 101 and a notifying section 102, as illustrated in FIG. 1.

The target detecting section 101 detects, based on a state of a member of a group, a target member in need of care provided by one or more other members of the group, the state being inferred from an image of the member. Note that in a case of inferring the state of each of a plurality of members of the group from an image of that member, the target detecting section 101 detects the target member from among the plurality of members. In a case of inferring the state of one member from an image of the one member, the target detecting section 101 judges whether the one member is the target member.

As used herein, the term “care” refers to actions in general which are performed in order to maintain or improve the state of a member. Examples of the “care” includes talking, by phone, to a member who is physically ill to check the state of the member, saying something and sending a message to a member who is feeling depressed or mentally unstable. The term “care” is interchangeable with consideration, attention, or help.

The state of a member in which the member is in need of the “care” may be defined in advance. For example, members such as a member with a high stress level inferred from an image and a member with a tendency toward a rise in stress level may be taken as the member in need of the “care”. In this case, the target detecting section 101 detects a target member based on the stress level of each member. Note that the stress level is an index value indicating the level of stress. For instance, a member whose stress level is equal to or greater than a predetermined threshold may be judged as being in a high-stress state and detected as the target member. Additionally, for example, a member whose stress level has increased a predetermined number of times may be identified based on past stress level records and detected as a member showing a tendency towards increasing stress. In this case, time-series stress levels for each member are obtained and used from a predetermined server or database (not illustrated).

The notifying section 102 notifies each member of the group of the target member detected by the target detecting section 101. The manner of the notification is not particularly limited. For example, the notifying section 102 may provide notification of the target member via terminals used by the respective members of the group, or may provide notification of the target member via displaying equipment (e.g. television) or the like shared by the respective members. Note that “each member”, who is a notification receiver, refers to other members, who are other than the target member. All of the other members, who are other than the target member, may be the notification receivers, or some of the other members may be the notification receivers.

As above, the information processing apparatus 1 includes: a target detecting section 101 for detecting, based on a state of a member of a group, a target member in need of care provided by one or more other members of the group, the state being inferred from an image of the member; and a notifying section 102 for notifying each member of the group of the target member detected. This configuration makes it possible to let each member know the target member in need of care provided by one or more other members, to promote mutual assistance among the members of the group. Accordingly, the information processing apparatus 1 provides an example advantage of making it possible to support mutual assistance among the members of a group. Alternatively, the information processing apparatus 1 makes it possible to optimize the manner of mutual assistance among the members of a group. Further, the information processing apparatus 1 also makes it possible to support decision-making in the mutual assistance among the members.

### [Configuration of Control Program]

The functions of the information processing apparatus 1 can also be implemented via a program. A control program in accordance with the present example embodiment causes a computer to function as: a target detecting means for detecting, based on the state of a member of a group, a target member in need of care provided by one or more other members of the group, the state being inferred from an image of the member; and a notifying means for notifying each member of the group of the target member detected. Thus, the control program in accordance with the present example embodiment provides an example advantage of making it possible to support mutual assistance among the members of a group.

### [Flow of Mutual Watching Method]

A flow of the mutual watching method will be described below with reference to FIG. 2. FIG. 2 is a flowchart illustrating a flow of the mutual watching method. It should be noted that each of the steps of this mutual watching method may be carried out by a processor included in the information processing apparatus 1, or may be carried out by a processor included in any other apparatus. Alternatively, the respective steps may be carried out by processors provided in different apparatuses.

In S1, at least one processor carries out a target detecting process of detecting, based on a state of a member of a group, a target member in need of care provided by one or more other members of the group, the state being inferred from an image of the member.

In S2, the at least one processor carries out a notifying process of notifying each member of the group of the target member detected in S1.

As above, in the mutual watching method, at least one processor carries out: a target detecting process of detecting, based on a state of a member of a group, a target member in need of care provided by one or more other members of the group, the state being inferred from an image of the member; and a notifying process of notifying each member of the group of the target member detected. Thus, the mutual watching method provides an example advantage of making it possible to support mutual assistance among the members of a group.

### Second Example Embodiment

The following description will discuss a second example embodiment, which is an example embodiment of the present invention, in detail with reference to the drawings. A component having the same function as a component described in the above example embodiment is assigned the same reference sign, and the description thereof is omitted where appropriate. It should be noted that the applicability of each of the technical means adopted in the present example embodiment is not limited to the present example embodiment. That is, each technical means adopted in the present example embodiment can be adopted in another example embodiment included in the present disclosure, to the extent of constituting no specific technical obstacle. Further, each technical means illustrated in the drawings referred to for the description of the present example embodiment can be adopted in another example embodiment included in the present disclosure, to the extent of constituting no specific technical obstacle.

### (Configuration of Mutual Watching System 3)

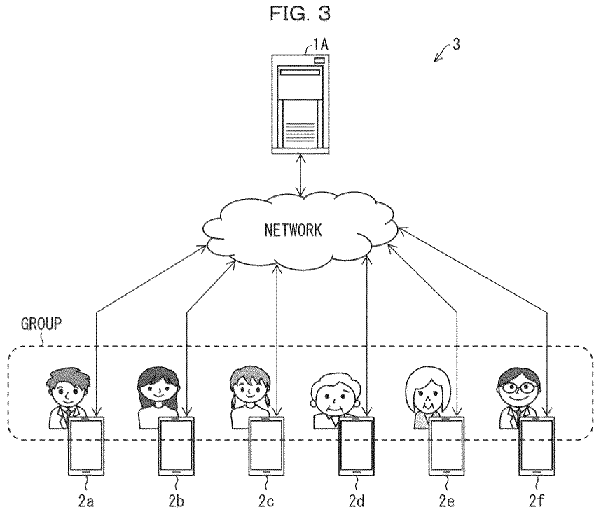

FIG. 3 is a representation of an example configuration of a mutual watching system 3. The mutual watching system 3 includes an information processing apparatus 1A and terminals 2a to 2f, as illustrated. The mutual watching system 3 supports mutual watching performed among the members of a group. For example, as illustrated, in a case where the respective users of the terminals 2a to 2f constitute a group, the information processing apparatus 1A supports mutual watching performed among the users. In the following description, the terminals 2a to 2f are denoted simply as “terminals 2” in a case where it is not necessary to distinguish therebetween. Although six terminals 2 are illustrated in FIG. 3, the mutual watching system 3 only needs to include at least two terminals 2.

The information processing apparatus 1A supports mutual watching performed among the members of a group. Like the information processing apparatus 1 described above, the information processing apparatus 1A detects, based on a state of a member of a group, a target member in need of care provided by one or more other members of the group, the state being inferred from an image of the member. The information processing apparatus 1A then notifies each member of the group of the target member detected.

The terminals 2 are used by the users of the mutual watching system 3, so that the users use the mutual watching system 3. For example, the terminals 2 display information which indicates the target member notified by the information processing apparatus 1A, to notify the users of the respective terminals 2 (i.e. members of the same group in which the target member is included) of the target member notified. Illustrated in FIG. 3 is the example in which the terminals 2 are smartphones. However, the terminals 2 only need to be equipment that implements the functions such as accepting a user's input, transmitting the accepted input to the information processing apparatus 1A, and presenting various kinds of information notified by the information processing apparatus 1A, and are not limited to smartphones. Further, the terminals 2 may each be portable equipment, or may each be stationary equipment.

As above, the mutual watching system 3 includes: an information processing apparatus 1A which detects, based on a state of a member of a group, a target member in need of care provided by one or more other members of the group, the state being inferred from an image of the member, and notifies each member of the group of the target member detected; and terminals 2 which present, based on the notification provided by the information processing apparatus 1A, the target member to the members who use the respective terminals 2. This provides an example advantage of making it possible to support mutual assistance among the members of a group.

In the mutual watching system 3, the members of a group are not assigned fixed roles. This makes it easy to maintain mutual assistance relationship even if the number of members increases or decreases. However, since some people are considered to think that they are in no need of care provided by the other members, whether the notification is necessary may be set on a member-by-member basis. Furthermore, since the target member is presented to each member of a group, it is possible to provide transparent operation.

Any members can constitute a group. For example, it is possible for the mutual watching system 3 to support mutual assistance among the members of a group constituted on a family-unit basis or a group constituted by various members of workers of the same work place, a sports team, various clubs, etc.

### [Configuration of Information Processing Apparatus 1A]

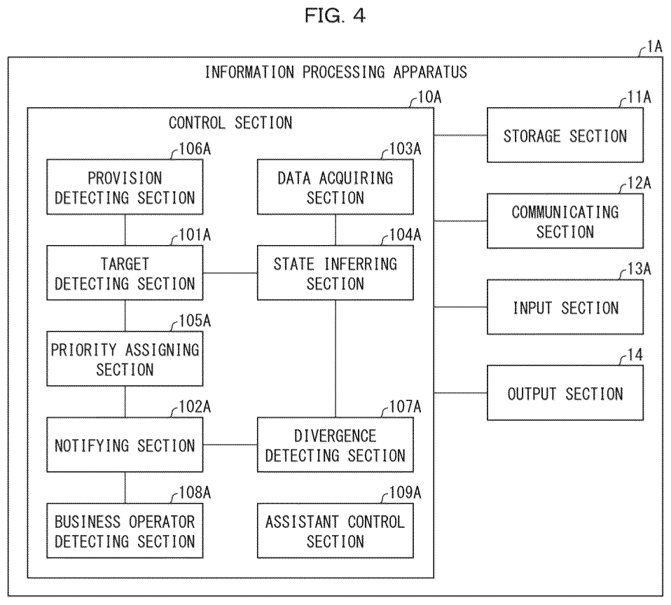

The configuration of the information processing apparatus 1A will be described below on the basis of FIG. 4. FIG. 4 is a block diagram illustrating an example configuration of the information processing apparatus 1A. As illustrated, the information processing apparatus 1A includes: a control section 10A for performing overall control of the sections of the generation apparatus 1A; and a storage section 11A in which various kinds of data used by the generation apparatus 1A are stored. The information processing apparatus 1A further includes: a communicating section 12A through which the information processing apparatus 1A communicates with another apparatus; an input section 13A for accepting the input, to the information processing apparatus 1A, of various kinds of data; and an output section 14 through which the information processing apparatus 1A outputs various kinds of data. Further, the control section 10A includes a target detecting section 101A, a notifying section 102A, a data acquiring section 103A, a state inferring section 104A, a priority assigning section 105A, a provision detecting section 106A, a divergence detecting section 107A, a business operator detecting section 108A, and an assistant control section 109A.

Like the target detecting section 101A illustrated in FIG. 1, the target detecting section 101A detects, based on the state of a member of a group, a target member in need of care provided by one or more other members of the group, the state being inferred from an image of the member. More specifically, in a case where the state of one member inferred by the state inferring section 104A (described later) meets a predetermined condition, the target detecting section 101A detects the one member as the target member.

The predetermined condition may be defined in advance according to the state inferred by the state inferring section 104A. Further, the predetermined condition may be defined for each member. For example, in a case where a member with hypertension is included in a group, what is inferred by the state inferring section 104A for the member and the predetermined condition for the member may be blood pressure and the blood pressure going out of the normal range, respectively. This makes it possible to, if the inferred value of the blood pressure of the member goes out of the normal range, notify other members of the blood pressure of the member going out of the normal range, to urge the other members to provide care.

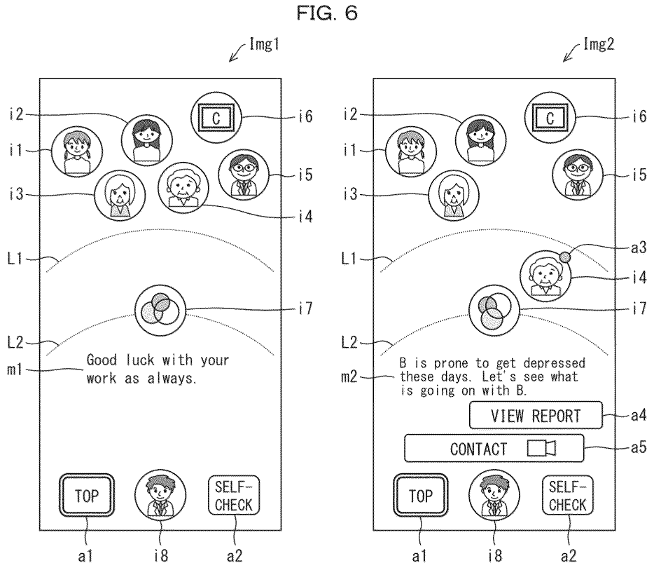

Like the notifying section 102 illustrated in FIG. 1, the notifying section 102A notifies a member of the target member detected by the target detecting section 101A, the member being each member of the same group in which the target member is included. Notification provided by the notifying section 102A will be described later on the basis of FIG. 6, etc.

The data acquiring section 103A acquires information required for inferring the state of a member. Specifically, the data acquiring section 103A acquires an image of a member of the group. For example, the data acquiring section 103A may acquire an image captured during a video call made by the member with use of the terminal 2 or the like and used for the video call.

The state inferring section 104A uses the image acquired by the data acquiring section 103A, to infer the state of the member shown in the image. The state to be inferred only needs to be a state which is capable of being inferred from an image and which serves as information for judging whether care provided by one or more other members is necessary. For example, the state inferring section 104A may infer at least one selected from the group consisting of stress level, degree of concentration, cognitive function, feelings, degree of arousal, and degree of tension. Further, in inferring these states, the state inferring section 104A may detect at least one selected from the group consisting of facial color change, facial movement, gaze, blink, pupil diameter, iris movement, and facial expression. As a method for inferring theses states from an image, well-known methods can be used.

The state inferring section 104A may further judge, based on the result of the above inference, disease or the presence or absence of a sign of the disease. For example, the state inferring section 104A may judge whether a member is showing a sign of at least one selected from the group consisting of dementia, mental disease, hypertension, heart disease, diabetes, and cancer. In a case of the judgment of disease, it is preferable to notify not only the members of a group but also medical personnel, such as a doctor.

The state inferring section 104A may use information other than an image, to infer the state of a member. For example, in a case where the member uses a wearable device, the state inferring section 104A may acquire various kinds of vital data and/or data on the amount of activity or the like that are measured by the wearable device, to use the data for the state inference. Further, the state inferring section 104A may infer the state in consideration of the history of disease, chronic disease, checkup result, etc. of the member.

The priority assigning section 105A assigns priorities which are used in the notifying section 102A providing notification of a target member. These priorities are for provision of care for a target member. That is, the notifying section 102A provides notification such that a member with a high priority assigned by the priority assigning section 105A preferentially provides care for a target member. As an example, the notifying section 102A may notify each of the members in the descending order of the priorities. As another example, the notifying section 102A may provide notification which includes a message in accordance with the priorities. For example, the notifying section 102A may provide notification of a message which includes information indicating the priorities, or may provide, to a member with a high priority, notification which includes a message strongly recommending providing care for a target member.

In a case where even if a predetermined amount of time passes after each of the members is notified of a target member, it cannot be confirmed that care for the target member has been provided, the notifying section 102A may provide reminder notification to each of the members. The notifying section 102A may determine, according to the priorities assigned by the priority assigning section 105A, whether to provide this reminder notification or the frequency of this reminder notification.

A priority assigning method is not particularly limited. As an example, the priority assigning section 105A may assign a priority which is designated by a member via the communicating section 12A or the input section 13A. As another example, the priority assigning section 105A may acquire the current situation (e.g. being on duty, off duty, etc.) of each member, to assign a priority according to the situation indicated by the information. This enables the notifying section 102A to cause notification to a member off duty to take precedence over to notification to a member on duty.

As still another example, the priority assigning section 105A may assign a priority based on the history of care provided by each of the members in the past. For example, the priority assigning section 105A may assign higher priority to a member who provided care a relatively small number of times or at a relatively low frequency in the past than to a member who provided care a relatively large number of times or at a relatively high frequency in the past. This also makes it possible to avoid the concentration of the burden of care on a specific member by preferentially notify a member who provided care a relatively small number of times or at a relatively low frequency in the past. As another example, the priority assigning section 105A may cause a member who provided effective care in the past to be preferentially notified, to increase the possibility that effective care is provided. If after one member provides care, there is an improvement in the state of a target member who receives the care, it is inferred that the care provided by the one member is effective. This enables the priority assigning section 105A to identify a member who has provided an effective care, by referring to the history of care and the post-care state of a target member.

As yet still another example, the priority assigning section 105A may assign a priority based on, for example, information on the position of each member, the relationship among members, the state inferred by the state inferring section 104A, and the characters of each member (an acquired qualification, age, gender, specialty, etc.). This makes it possible to preferentially provide notification to a member such as a member located close to a target member, a member friendly with a target member, or a member capable of providing care according to the state of a target member.

In this manner, the information processing apparatus 1A may include the priority assigning section 105A for assigning a priority to each of the other members, who are other than a target member, in terms of providing care for the target member. The notifying section 102A may then notify, based on the priority, each member of the group. This provides an example advantage of making it possible to provide appropriate notification in consideration of differences between members, in addition to the example advantage provided by the information processing apparatus 1.

The provision detecting section 106A detects provision, by a member of the group, of care for a target member. A method for detecting provision of care is not particularly limited. As an example, in a case where notification to the effect that care has been provided is received from a member who provided the care or the target member who received the care, the provision detecting section 106A may detect the provision of care. As another example, care can be provided by sending a message to a target member via the terminal 2, or by making a video call or a voice call to a target member via the terminal 2. In such a case, the provision detecting section 106A may detect the provision of care in a case where a message to a target member is sent or a call with a target member is made.

In this manner, the information processing apparatus 1A may include the provision detecting section 106A for detecting provision, by a member of a group, of care for a target member. The notifying section 102A may then end notification regarding the target member on condition that provision of care for the target member is detected. This provides an example advantage of making it possible to let each member know that care for a target member has been provided and other members therefore no longer need to provide care, in addition to the example advantage provided by the information processing apparatus 1.

The divergence detecting section 107A detects a divergence between a result of self-evaluation, performed by a member, of the state of the member and the state of the member inferred by the state inferring section 104A. The result of self-evaluation performed by a member may be inputted by the member via, for example, the terminal 2. Further, based on what relationship between the result of self-evaluation and an inferred state it is judged that there is a divergence between the result and the inferred state may be defined in advance. For example, in a case where the state inferring section 104A evaluates a stress level by five-grade evaluation on a scale of 1 to 5, the self-evaluation of a stress level may also be performed by five-grade evaluation. In this case, in a case where, for example, there is a difference between the grades of the stress level, the divergence detecting section 107A may detect a divergence between the grades.

The business operator detecting section 108A detects a business operator pertaining to care for a target member, from among the business operators included in the members of a group. As used herein, the “business operator” covers individuals and corporates in general which do business. In this manner, registering a business operator as a member of a group may be allowed in the mutual watching system 3. For example, by registering, as a member of a group, a temporary care worker employment agency, a hospital, a doctor, a health professional, or an insurance company, it is possible to provide watching among members which include these business operators or employees of the business operators.

The business operator detecting section 108A may detect a business operator pertaining to care for a target member, based on, for example, at least one selected from the group consisting of a target member and the state of the target member. As an example, in a case where the target member is an elderly person, the business operator detecting section 108A may detect a business operator pertaining to nursing care, from among the business operators registered as members of the group. As another example, in a case where a target member is in such a state as to require treatment, the business operator detecting section 108A may detect a business operator pertaining to medical care, from among the business operators registered as members of the group.

The assistant control section 109A is for assisting in using the mutual watching system 3. More specifically, the assistant control section 109A controls the action of a virtually existing assistant (hereinafter referred to as a virtual assistant) who interacts with a member using the mutual watching system 3, to assist a member in using the mutual watching system 3.

For example, the assistant control section 109A may assist a member, by inputting a query to a generative artificial intelligence (AI) to cause the generative AI to generate a message, and presenting the message to the member by, for example, displaying the generated message on the terminal 2. The generative AI to be used may be, for example, a language model having learned, by machine learning, the arrangement of the components (such as words) of a sentence and the arrangement of sentences in text. By using such a language model, the assistant control section 109A can generate a message to be presented to a member and generate an answer to a question inputted by a member.

### [Configuration of Terminals 2]

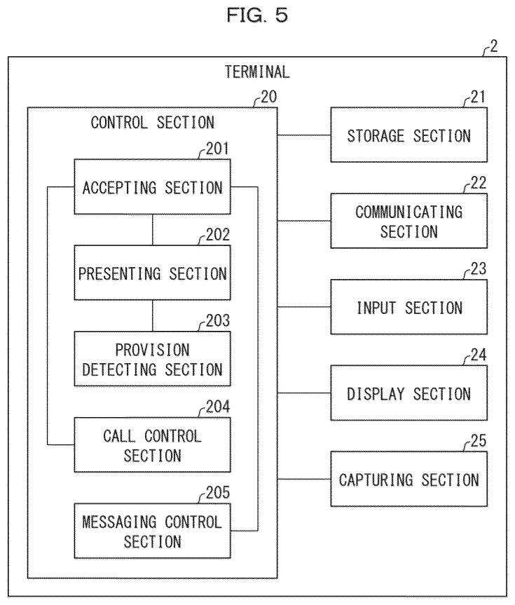

The configuration of the terminals 2 will be described below on the basis of FIG. 5. FIG. 5 is a block diagram illustrating an example configuration of the terminals 2. As illustrated, the terminals 2 each include: a control section 20 for performing overall control of the sections of the terminal 2; and a storage section 21 in which various kinds of data used by the terminal 2 are stored. Further, the terminals 2 each include: a communicating section 22 via which the terminal 2 communicates with another apparatus; an input section 23 for accepting the input, to the terminal 2, of various kinds of data; a display section 24 for displaying an image; and a capturing section 25 for capturing an image (still image or moving image). Further, the control section 20 includes an accepting section 201, a presenting section 202, a provision detecting section 203, a call control section 204, and a messaging control section 205. For example, by installing on a general-purpose computer a control program for causing a computer to function as each of these sections, it is possible to cause the computer to function as the terminal 2.

The accepting section 201 accepts various inputs regarding the mutual watching system 3. For example, the accepting section 201 accepts an input from a member who is using the terminal 2, to notify the information processing apparatus 1A of the content of the input. As another example, the accepting section 201 accepts various notifications (e.g., notification of a target member) from the information processing apparatus 1A.

The presenting section 202 presents various kinds of information regarding the mutual watching system 3. For example, in a case where the accepting section 201 accepts notification of a target member, the presenting section 202 may light an indicator (not illustrated) of the terminal 2 or cause an audio output section (not illustrated) to output a notification sound, to notify the member who is using their own terminal 2 of reception of the notification.

The presenting section 202 displays various UI screens on the display section 24, to present various kinds of information regarding the mutual watching system 3, to a member who is using the terminal 2. The UI screens may be generated by the information processing apparatus 1A and then acquired and displayed by the presenting section 202, or may be generated by the presenting section 202. The UI screens will be described later on the basis of FIGS. 6 to 9.

The provision detecting section 203 detects provision, by a member who is using the terminal 2, of care for a target member. Further, upon detection of provision of care for a target member, the provision detecting section 203 notifies the information processing apparatus 1A of the detection. A method for the detection is not particularly limited. As an example, in a case where a call with a target member is made via the call control section 204 (described below) or a message to a target member is s transmitted via the messaging control section 205 (described below), the provision detecting section 203 may detect provision of care for the target member. As another example, in a case where the accepting section 201 accepts an input operation which indicates that care for a target member has been provided, the provision detecting section 203 may detect the provision of care for the target member.

The call control section 204 performs control for making a video call among members. More specifically, the call control section 204 originates a video call to a member according to the input accepted by the accepting section 201. Further, the call control section 204 carries out the process of terminating a video call and the process of switching the input and output of voice to a speaker mode (hands-free call mode), according to the input accepted by the accepting section 201. The call control section 204 can cause a voice-only call, in which no video is displayed, to be made.

The messaging control section 205 performs control for exchanging messages among members. More specifically, the messaging control section 205 carries out processes such as the process of displaying, on a timeline, messages transmitted and received among the members in the past, the process of accepting the input of a new message, and the process of adding an inputted message to a timeline. Note that the messages are not limited to those to be displayed on a timeline. For example, the messaging control section 205 may notifies each of the members of a message for that member.

### [Example User Interface (UI) Screens: Notification of Target Member]

Example UI screens displayed on the terminals 2 in the mutual watching system 3 will be described below on the basis of FIG. 6. FIG. 6 is a representation of example UI screens displayed before and after the notification of a target member. Specifically, in FIG. 6, an Img 1 is an example UI screen on which notification of a target member is not being provided, and an Img 2 is an example UI screen on which notification of a target member is being provided.

A member who uses the mutual watching system 3 performs a login to the mutual watching system 3 by, for example, inputting the identification (ID) of the member into the terminal 2. The presenting section 202 identifies the member on the basis of the ID, and displays on the display section 24 a UI screen for the member. The Img 1 and the Img 2 are examples of the UI screen, displayed in such a manner, for an individual member. The process of generating the Img 1 and the Img 2 may be carried out by the information processing apparatus 1A, or may be carried out by the terminal 2. In the following description, a member who browses the UI screen after performing the login as described above is referred to as a browsing member.

In the Img 1, eight icons which are i1 to i8 are displayed. Among these icons, i8 is the icon of the browsing member. Further, i7 is the icon indicating the virtual assistant described above. Furthermore, i2 to i6 are the icons of the respective members who belong to the same group as the browsing member. More specifically, among the icons i2 to i6, the icons i2 to i5 indicate persons each registered as the member, and the icon i6 indicates a business operator registered as the member. For each of the icons i2 to i5 and i8, the image of the corresponding member is displayed, whereas for the icon i6, a mark indicating a business operator is displayed. In this manner, the presenting section 202 of the terminal 2 may display the icon of a business operator and the icon of a person in an identifiable manner.

In the Img 1, the icons i2 to i6 of the members are displayed in an at-a-glance manner above a line L1. The icon i8 of the browsing member and a message m1 are displayed below a line L2. The message m1 is a message generated by a generative AI and meant for the browsing member, and is displayed as a message from the virtual assistant.

In the Img 1, the icon i7 is disposed between the icon i1 and the icons i2 to i6. This makes it possible to make the browsing member aware that the virtual assistant represented by the icon i7 is an intermediate between the browsing member and the members of the group which are represented by the icons i2 to i6.

In the Img 1, a button a1 for displaying a top screen and a button a2 for initiating a self-check are also displayed. Note that the top screen is a screen of the Img 1 in which the icons of the respective members are displayed in an at-a-glance manner.

The self-check means the browsing member causes the information processing apparatus 1A to judge their own state. Upon acceptance of an operation performed on the button a2, the accepting section 201 activates the capturing section 25 to capture an image of a member, and transmits the captured image to the information processing apparatus 1A. Upon reception of the image, in the information processing apparatus 1A, the data acquiring section 103A acquires the image, and the state inferring section 104A analyzes the image to infer the state of the browsing member. The terminal 2 is notified of the result of the inference, and the presenting section 202 of the terminal 2 presents the notified result of the inference to the browsing member, by, for example, displaying the notified result on the display section 24.

In the Img 2 illustrated in FIG. 6, among the icons i2 to i6 of the members, the display position of the icon i4 is changed to the area between the lines L1 and L2. The icon i4 represents the target member detected by the target detecting section 101A of the information processing apparatus 1A. Such a change in the display position is performed on the basis of the notification from the notifying section 102A.

In this manner, the presenting section 202 may present a target member to the browsing member, on the basis of the notification from the notifying section 102A, by changing the display position of the icon i4 of the target member from the original display position, which is the area above the line L1, to a position located within a predetermined display area sandwiched between the lines L1 and L2. This makes it possible to make the browsing member intuitively aware that a target member is in need of care.

A badge a3 is displayed so as to be associated with the icon i4. This makes it possible to make the browsing member aware that there is information that should be checked out regarding the target member corresponding to the icon a4. For example, the presenting section 202 may end the display of the badge a3 in response to the browsing member having checked out a report on the state of the target member.

In the Img 2, a message m2 describing the state of the target member (named B) is also displayed. The message m2 may be generated by putting the name of the target member into a state-specific template prepared in advance. Alternatively, the assistant control section 109A may cause a generative AI to generate the message m2. That is, the notifying section 102A may provide notification of a message which is generated with use of a generative AI, i.e. a language model trained by machine learning and which describes the state of the target member.

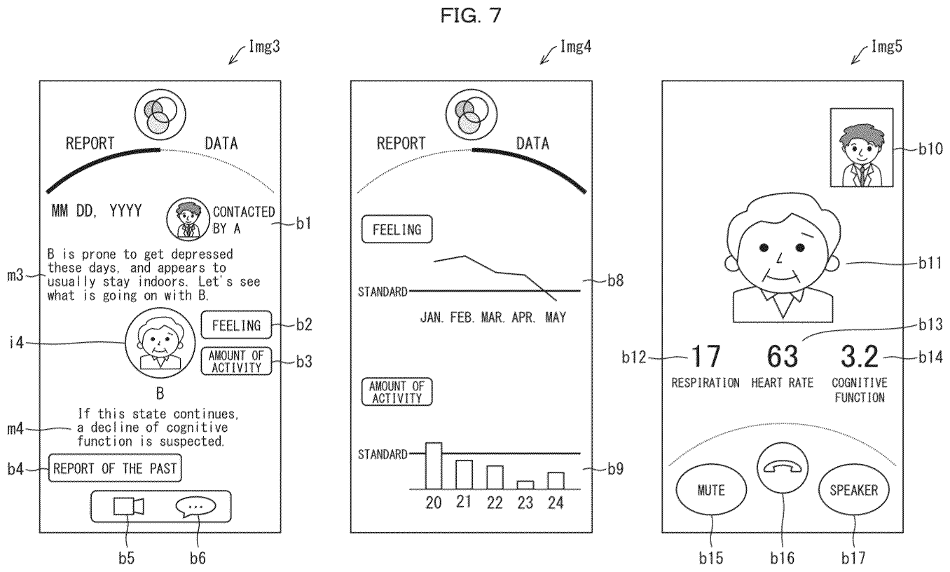

In the Img 2, a button a4 for displaying a report on the analysis of the state of the target member is also displayed. In a case where the browsing member performs an input operation on the button a4, the presenting section 202 displays the report on the display section 24. The details of the report will be described later on the basis of FIG. 7.

In the Img 2, a button a5 for making a video call with the target member is also displayed. In a case where the browsing member performs an input operation on the button a5, the call control section 204 originates a call for making a video call with the target member. This enables the browsing member to smoothly start a video call with the target member.

As above, from among icons which correspond to the respective members of the group and which are displayed on a display screen that the members are allowed to browse, the notifying section 102A may change the display position of the icon of the target member to a position within a predetermined display area set on the display screen. This provides an example advantage of making it possible to make each member of a group easily aware of the emergence of a member who becomes the target member among the members, in addition to the example advantage provided by the information processing apparatus 1.

Further, as described above, the notifying section 102A may provide notification of a message which is generated with use of a language model trained by machine learning and which describes the state of the target member. This provides an example advantage of making it possible to pass the state of a target member to other members in a comprehensible manner, in addition to the example advantage provided by the information processing apparatus 1.

Any area can be the determined display area. For example, the predetermined display area may be set between the area in which to display the icons of the members and the area in which to display the icon of the browsing member, as in the example UI screens illustrated in FIG. 6. This makes it possible to make the browsing member feel as if the target member asks the browsing member for care, and thereby urge the browsing member to provide care.

The notifying section 102A may generate a UI screen having the display position of the icon of the target member changed therein and transmit the generated UI screen to the terminal 2, to cause the terminal 2 to display the UI screen. Further, the UI screen may be generated by the presenting section 202 of the terminal 2. In this case, from among the icons which correspond to the respective members of a group and which are displayed on a display screen that the members are allowed to browse, the presenting section 202 changes the display position of the icon of the target member to a position within the predetermined display area set on the display screen, according to the notification from the notifying section 102A.

In a case where the provision detecting section 106A detects the provision, by a member of the group, of care for the target member, the notifying section 102A ends the notification regarding the target member. For example, in a case where provision of care for the target member is detected while the UI screen is displayed, the notifying section 102A may generate a new UI screen and transmit the generated UI screen to the terminal 2, to cause the UI screen to be updated. In this respect, the new UI screen is the UI screen having the display position of the icon of the target member returned to the display area in which to display the icons of the respective members (the area above the line L1 in the example of FIG. 6).

As to the members for which the UI screen is not displayed at the time of detection of the provision of care for the target member, the notifying section 102A causes a UI screen to be displayed for such members, the UI screen having the display position of the icon of the target member returned to the display area in which to display the icons of the respective members of the group, at the next UI screen display timing or any subsequent UI screen display timing. The update and/or generation of such a UI screen may be carried out by the presenting section 202 of the terminal 2.

### [Example UI Screens: Report⋅Data⋅Video Call]

FIG. 7 is a representation of example UI screens displayed during presentation of a report, presentation of data, and a video call. Specifically, in FIG. 7, an Img 3 is an example UI screen on which to present a report, an Img 4 is an example UI screen on which to present data which supports the report, and an Img 5 is an example UI screen displayed during a video call.

The Img 3 indicates a report on the state of the target member named B. In the Img 3, not only the icon i4 of B is displayed, but a message m3 describing the state of B, a message m4 describing matters to be attended to regarding the state of B, and objects b2 and b3 representing pieces data which support the detection of B as the target member are also displayed. The messages m3 and m4 may be in fixed forms which are in accordance with the state of the target member, or the assistant control section 109A may cause a generative AI to generate the messages m3 and m4.

In the Img 3, also displayed are: the date of preparation of the report; information b1 indicating a member who has contacted B; a button b4 for displaying a report of the past; a button b5 for making a video call with B; and a button b6 for transmitting a message to B.

In a case where the button b4 is operated, the presenting section 202 displays a report of the past. In a case where the button b5 is operated, the call control section 204 originates a call for making a video call with the target member. In a case where the button b6 is operated, the messaging control section 205 accepts input of a message meant for the target member by, for example, displaying an input acceptance screen on which a message to the target member is accepted.

The Img 4 illustrates an example UI screen for presenting data which supports the report of the Img 3. Specifically, displayed in the Img 4 are: a graph representing a change over time in “feeling” indicated in the object b2 of the Img 3; and a graph representing a change over time in the “amount of activity” indicated in the object b3 of the Img 3. The “feeling” and the “amount of activity” are inferred by the state inferring section 104A. By storing, in the storage section 11A or the like, the results of inference carried out by the state inferring section 104A, the notifying section 102A can use the stored results of inference to generate a UI screen which contains such graphs. Optionally, the notifying section 102A may notify the terminal 2 of the results of inference, and the presenting section 202 of the terminal 2 may generate, on the basis of the notified results of inference, a UI screen which contains the graphs.

The Img 5 illustrates an example UI screen during a video call. In the Img 5, displayed are: an image b10 of the browsing member; an image b11 of the other end (the target member in this example) of the call; pieces of information b12 to b14 which indicate the results of state inference carried out by the state inferring section 104A; a mute button b15 for muting utterance given by the browsing member; a call end button b16 for ending a video call; and a switch button b17 for switching audio input-output to a speaker mode.

The data acquiring section 103A may acquire time series images (which may be a moving image) captured during a video call and used for the video call. The state inferring section 104A may then infer the state of a member from each of the acquired images (or each of the frame images extracted from a moving image). This enables the notifying section 102A to update, in real time, the pieces of information b12 to b14 displayed on the UI screen.

As above, the state inferring section 104A may infer the state of a member from images captured during a video call made by the member and used for the video call. This eliminates the need to cause the member to capture images for the state inference. Further, for example, simply by making regular video calls among the members, it is possible to record changes in the state.

As described above, the state inferring section 104A may repeatedly carry out state inference during a video call, to generate a time-series state inference result. In this case, the target detecting section 101A may use a representative value of the time-series inference result provided by the state inferring section 104A, to judge whether the member is right for the target member. Examples of the representative value include a mean, a maximum, a minimum, a median, and a mode.

The state inference results or the like are private information regarding the members. As such, the members may each set their respective scopes of disclosure and their respective pieces of information subject to disclosure. In this case, the notifying section 102A or the presenting section 202 provides notification or presentation of the state inference results or the like within the set scope.

### [Example UI Screens: Presentation of Business Operator]

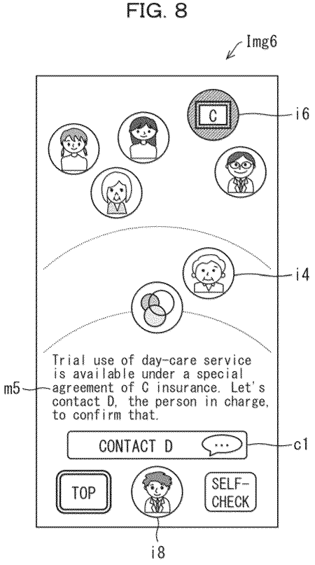

FIG. 8 is a representation of an example UI screen on which business operators pertaining to care for a target member are presented. For example, an Img 6 illustrated in FIG. 8 is displayed after the browsing member (the member represented by the icon i8) to which the UI screen such as the Img 2 of FIG. 6 is presented provides care for the target member (the member represented by the icon i4).

The Img 6 differs from the Img 2 of FIG. 6 in that: the display manner of the icon i6 is changed; the badge a3 displayed in association with the icon i4 disappears; the message is changed from m2 to m5; and a button c1 is displayed instead of the buttons a4 and a5.

The icon i6 is the icon representing a business operator pertaining to care for the target member, i.e. the business operator detected by the business operator detecting section 108A. In the Img 6, a business operator corresponding to the icon i6 is an insurance company named “C insurance”, and the target member B has an insurance policy with this insurance company. As in the example of FIG. 8, the notifying section 102A may change the display manner of an icon representing the business detected by the business operator detecting section 108A, to notify the browsing member of the business operator. Note that the display manner of an icon is changed to any manner.

A message m5 is a message regarding the business operator detected by the business operator detecting section 108A. Specifically, the message m5 is a message by which to notify that there is a service available under a special agreement of the insurance held by the target member named B and to urge the browsing member to contact a person (named D) in charge of the insurance. In this manner, the notifying section 102A may notify the browsing member of information (in particular, information useful for care for the target member) on the business operator detected by the business operator detecting section 108A. Thus, it is possible to support the browsing member in decision-making regarding early handling of B.

The button c1 is a button for transmitting a message to the business operator (more properly, a person in charge in the business operator) detected by the business operator detecting section 108A. This enables the browsing member to smoothly contact the business operator. The notifying section 102A may also display a button for starting to make a call (which may be a video call, or may be a voice-only call) with a business operator or a person in charge of the business operator.

As described above, a business operator may be included in a group as a member of the group. Further, the information processing apparatus 1A may include the business operator detecting section 108A for detecting a business operator pertaining to care for the target member, from among the business operators included in the members. The notifying section 102A may then notify the member who has provided care for the target member, of the detected business operator. This provides an example advantage of making it possible to make it easy to use a business operator in providing care for the target member, in addition to the example advantage provided by the information processing apparatus 1.

[Example UI Screens: Notification Regarding Divergence from Self-Evaluation]

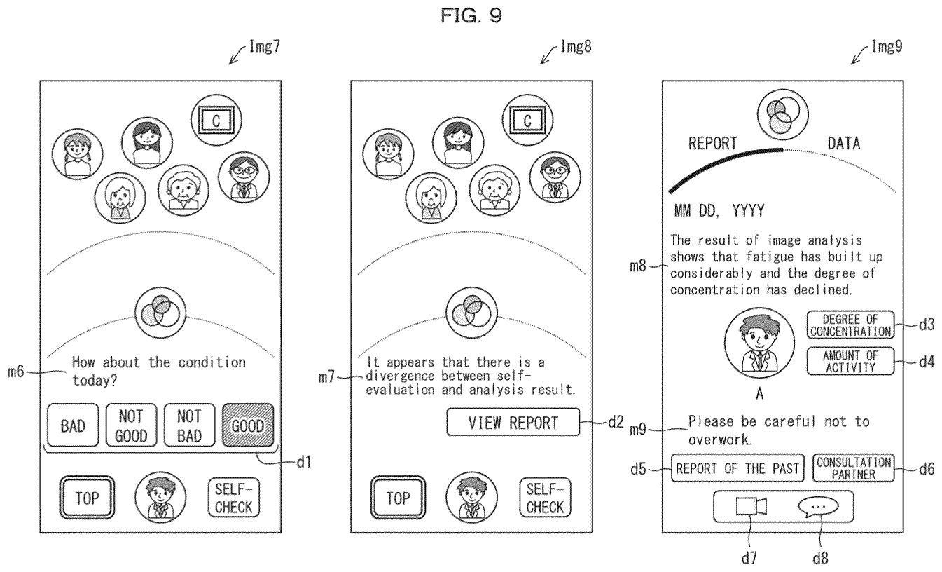

The divergence detecting section 107A detects a divergence between a result of self-evaluation, performed by a member, of the state of the member and the state of the member inferred by the state inferring section 104A. The notifying section 102A also provides notification to the member for which the divergence is detected, the notification urging the member to be careful. A specific example of this notification will be described below on the basis of FIG. 9.

FIG. 9 is a representation of an example UI screen on which a divergence between the result of self-evaluation of a state and the result of inference of the state is notified. More specifically, an Img 8 of FIG. 9 illustrates an example UI screen for divergence notification. An Img 7 is an example UI screen for self-evaluation of a state, and an Img 9 is an example UI screen for presenting a report on a state inference result.

In the Img 7, a message m6 for asking the state of the browsing member is displayed, and candidates d1 for the state is also displayed. The candidates d1 includes four levels from “bad” to “good”. The browsing member can input the result of self-evaluation by selecting a candidate close to the state that the browsing member is aware of, from among the candidates d1. In this manner, the notifying section 102A may present a plurality of candidates for a state, to the browsing member. In this case, the divergence detecting section 107A can identify the result of the self-evaluation performed by the browsing member, on the basis of the result of the selection made by the browsing member. In the example of the Img 7, the candidate “good” is highlighted. This indicates that the candidate “good” is selected, i.e. the result of self-evaluation performed by the browsing member is that the state of the browsing member is good.

The message m6 may be a fixed message, or the assistant control section 109A may cause a language model to generate the message. Further, after self-evaluation is performed, the notifying section 102A may urge the browsing member to capture an image of the browsing member, the data acquiring section 103A may acquire the captured image, and the state inferring section 104A may use the acquired image to infer the state of the browsing member. This enables the divergence detecting section 107A to use the result of state inference carried out at the time of self-evaluation, to detect a divergence. In a case of detection of a divergence, the notifying section 102A urges the browsing member to be careful by, for example, displaying a UI screen such as the Img 8.

In the Img 8, a message m7 which indicates that there is a divergence between the result of self-evaluation of a state and the result of analysis of the state, i.e. the result of inference carried out by the state inferring section 104A is displayed. The message m7 may be a fixed message, or may be the assistant control section 109A may cause a language model to generate the message. In the latter case, the assistant control section 109A may generate a query instructing that a message for notification of the divergence between the self-evaluation and the analysis result be created, and input the query to a language model.

In the Img 8, a button d2 for displaying a report on the result of state inference is displayed. Upon performance, by the browsing member, of an input operation on the button d2, a UI screen such as, for example, the Img 9 is displayed.

The Img 9 illustrates a report on the state of the browsing member named A. In the Img 9, a message m8 describing the state of A and a message m9 describing matters to be attended to regarding the state of A are displayed. As these messages, messages prepared in advance in accordance with the state, the degree of divergence, etc. may be displayed, or the assistant control section 109A may cause a language model to generate the messages.

In the Img 9, objects d3 and d4 which indicate data based on which A is detected as the target member are displayed. Also in the Img 9, a button d5 for displaying a report of the past, a button d6 for consulting a predetermined consultation n partner, a button d7 for making a video call with the predetermined consultation partner, and a button d8 for transmitting a message to the predetermined consultation partner are displayed.

The predetermined consultation partner may be determined in advance, or may be determined according to the browsing member and the state of the browsing member. As an example, the notifying section 102A may present, as the consultation partner, a member who has interacted (made calls or exchanged messages) with the browsing member at a high frequency, from among the members of the group. As another example, the notifying section 102A may present, as the consultation partner, a business operator (e.g. a regular doctor, etc.) pertaining to the state of the browsing member, from among the business operators registered as members of the group.

As above, the information processing apparatus 1A includes the divergence detecting section 107A for detecting a divergence between the result of self-evaluation, performed by a member, of the state of the member and the state of the member inferred from an image. The notifying section 102A then provides notification to the member for which the divergence is detected, the notification urging the member to be careful. This provides an example advantage of making it possible to make a member aware that the member might be in such a dangerous state as to be incapable of performing a proper self-evaluation, in addition to the example advantage provided by the information processing apparatus 1. The notifying section 102A may also notify each member of a group of the target member who is the member for which a divergence is detected. This makes it possible to cause the entire group to be involved in care for the member who might be in such a dangerous state as to be incapable of performing a proper self-evaluation.

### [Flow of Processes: Information Processing Apparatus 1A]

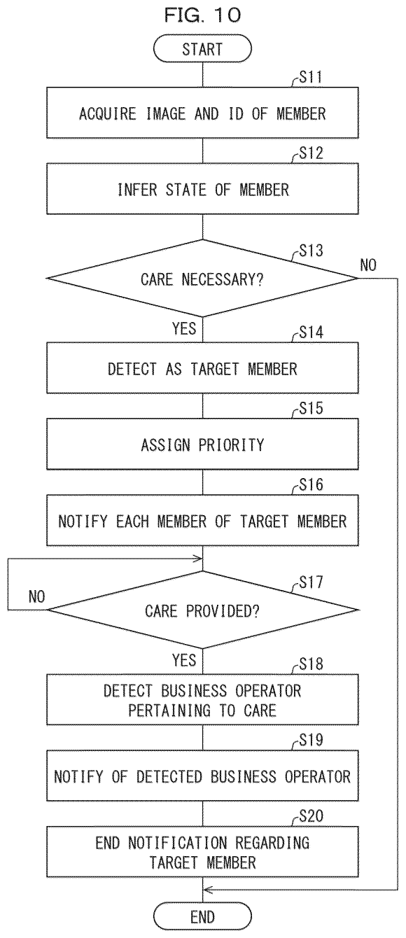

A flow of processes carried out by the information processing apparatus 1A will be described below on the basis of FIG. 10. FIG. 10 is a flowchart illustrating a flow of processes carried out by the information processing apparatus 1A. The flow of FIG. 10 includes the mutual watching method in accordance with the present example embodiment.

In S11, the data acquiring section 103A acquires the image of a member. For example, the data acquiring section 103A may acquire an image captured by the terminal 2 during a video call made by the member, or may acquire an image captured by the terminal 2 during a self-check performed by the member. Further, the data acquiring section 103A acquires the ID of the member, in addition to the image of the member. The data acquiring section 103A may also acquire data useable in inferring the state of the member, such as vital data of the member.

In S12, the state inferring section 104A uses the image acquired in S11, to infer the state of the member shown in the image. Note that in a case where data other than the image is acquired in S11, the state inferring section 104A also uses the data to infer the state of the member.

In S13, based on the result of the inference carried out in S12, the target detecting section 101A judges whether care for the member subjected to state inference is necessary. As described above, based on which state of a member it is judged that care is necessary is defined in advance. In a case of No judgment in S13, the processing of FIG. 10 ends. In a case of YES judgment in S13, the processing continues to S14.

In S14, the target detecting section 101A detects, as the target member, a member shown in the image acquired in S11. In this manner, the target detecting section 101A carries out a target detecting process of detecting, based on the state of a member of a group, a target member in need of care provided by one or more other members of the group, the state being inferred from the image of the member.

In S15, the priority assigning section 105A assigns, for each member of the same group that the target member detected in S14 is included, a priority in terms of providing care for the target member. The method for assigning the priority is as described above, and the description thereof is therefore omitted here.

In S16, the notifying section 102A notifies each member of the same group that the target member detected in S14 is included, of the target member, according to the priority assigned in S15. In this manner, the notifying section 102A carries out a notifying process of notifying each member of the above group of the target member detected by the target detecting section 101A. The notification may contain the ID of the target member. Further, as described above, the notification may be provided in the descending order of the priority assigned by the priority assigning section 105A.

In S17, the provision detecting section 106A judges whether care for the target member has been provided by a member of the group. In a case of NO judgment in S17, the process of S17 is carried out again after the elapse of a predetermined amount of time. In a case of YES judgment in S17, the processing continues to S18. Note that in a case where the number of repetitions of the process of S17 reaches a predetermined upper limit, the processing of FIG. 10 may be ended, or the notifying section 102A may provide notification again.

In S18, the business operator detecting section 108A detects a business operator pertaining to care for the target member, from among the business operators included as members in the same group that the target member is included. Further, in S19, the notifying section 102A notifies the member who has provided care for the target member, of the business operator detected in S18. Note that the notifying section 102A may notify the other members of the business operator. In a case where no business operator pertaining to care for the target member is registered, the processes of S18 and S19 are omitted.

In S20, the notifying section 102A ends the notification pertaining to the target member detected in S14, and the processing of FIG. 10 ends accordingly. For example, in a case where a UI screen such as the Img 2 of FIG. 6 generated by the notifying section 102A is displayed, the notifying section 102A performs an update to change this UI screen to a UI screen such as the Img 1. In a case where such a UI screen is generated by the presenting section 202 of the terminal 2, the notifying section 102A transmits the ID of the target member to the terminal 2, and instructs that the display position of the icon of a member identified by the ID be changed.

### [Flow of Processes: Terminal 2]

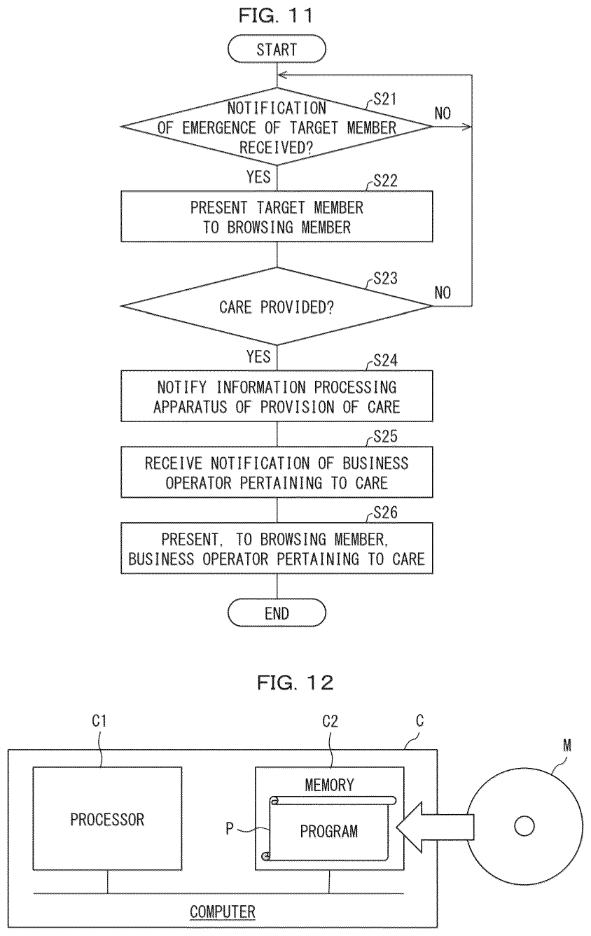

A flow of processes carried out by the terminal 2 will be described below on the basis of FIG. 11. FIG. 11 is a flowchart illustrating a flow of processes carried out by the terminal 2.

In S21, the accepting section 201 judges whether notification indicating the emergence of a target member is received. This notification is transmitted from the information processing apparatus 1A in S16 of FIG. 10. In a case of YES judgment in S21, the processing continues to S22, and in a case of NO judgment in S21, the judgment of S21 is carried out again after the elapse of a predetermined amount of time.

In S22, on the basis of the notification received in S21, the presenting section 202 presents the target member to the member who uses the terminal 2. The presentation is performed in any manner. For example, in a case where the member who is using the terminal 2 is browsing the UI screen of the mutual watching system 3 (i.e. in a case where this member is the browsing member), the presenting section 202 may present the target member by changing the display position of the icon of the target member (see the Img 2 of FIG. 6).

In a case where the above notification is received in a situation where the UI screen is not displayed, the presenting section 202 may first urge the member to check the UI screen, and present the target member by displaying a UI screen such as the Img 2 in response to the operation of displaying the UI screen. A method for urging the member to check the UI screen is not particularly limited. For example, the presenting section 202 may urge the member to check the UI screen by causing the terminal 2 to produce at least one selected from the group consisting of light, sound, and vibration, or by displaying a message or an object on the display section 24.

In S23, the provision detecting section 203 judges whether care has been provided by the browsing member, who is browsing the above UI screen. For example, in a case where a UI screen such as the Img 2 of FIG. 6 is presented to the browsing member, and the operation of selecting the button a5 is performed, a video call between the browsing member and the target member is made via the call control section 204. In a case where such a call has been made or in a case where a message has been transmitted from the browsing member to the target member via the messaging control section 205, the provision detecting section 203 may judge that care has been provided by the browsing member. In a case of NO judgment in S23, the processing returns to S21. In a case of YES judgment in S23, the processing continues to S24.

In S24, the provision detecting section 203 notifies the information processing apparatus 1A that the browsing member has provided care for the target member. This notification may contain the ID of the browsing member and the ID of the target member. Upon reception of this notification, in the information processing apparatus 1A, YES judgment is made in S17 of FIG. 10, and the processes of S18 and S19 are carried out. Accordingly, the terminal 2 is notified of the business operator pertaining to care for the target member.

In S25, the accepting section 201 receives the above notification, which indicates the business operator pertaining to care for the target member. In a case where there is no business operator detected by the information processing apparatus 1A, the processes of S25 and S26 are not carried out, and the illustrated processing ends.

In S26, the presenting section 202 presents to the browsing member the business operator indicated in the notification received in S25. With this, the processing of FIG. 11 ends. For example, the presenting section 202 may present a business operator by highlighting the icon of the business operator on the UI screen, as in the example of the Img 6 of FIG. 8. The presenting section 202 may also display on the UI screen, for example, a message regarding the business operator, and a button for contacting the business operator or a person in charge of the business operator.

### Variation

The performer of each of the processes described in the above example embodiments is any performer, and is not limited to the above examples. That is, apparatuses which constitute the mutual watching system 3 can be changed as appropriate provided that the apparatuses are capable of carrying out the processes described in the above example embodiments. For example, one or more of the functions of the information processing apparatus 1A may be implemented by another server, or one or more of the functions of the information processing apparatus 1A may be implemented by the terminal 2. In the flowcharts illustrated in FIGS. 10 and 11, the processes of the steps may be carried out by a single apparatus (interchangeable with a processor), or may be carried out by respective apparatuses (similarly, interchangeable with processors). That is, the processes illustrated in FIGS. 10 and 11 may be carried out by a single processor, or may be carried out by a plurality of processors.

### Software Implementation Example

Some or all of the functions of each of the information processing apparatuses 1 and 1A and terminal 2 may be implemented by hardware such as an integrated circuit (IC chip), or may be implemented by software.

In the latter case, the information processing apparatuses 1 and 1A and the terminal 2 are provided by, for example, a computer that executes instructions of a program that is software implementing the foregoing functions. An example (hereinafter, computer C) of such a computer is illustrated in FIG. 12. FIG. 12 is a block diagram illustrating a hardware configuration of the computer C which functions as the information processing apparatuses 1 and 1A and the terminal 2.

The computer C includes at least one processor C1 and at least one memory C2. The memory C2 has recorded thereon a program P (control program) for causing the computer C to operate as the information processing apparatuses 1 and 1A and the terminal 2. The processor C1 of the computer C retrieves the program P from the memory C2 and executes the program P, so that the functions of the information processing apparatuses 1 and 1A and the terminal 2 are implemented.

Examples of the processor C1 can include a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, and a combination thereof. Examples of the memory C2 can include a flash memory, a hard disk drive (HDD), a solid state drive (SSD), and a combination thereof.

The computer C may further include a random access memory (RAM) into which the program P is loaded at the time of execution and in which various kinds of data are temporarily stored. The computer C may further include a communication interface via which data is transmitted to and received from another apparatus. The computer C may further include an input-output interface via which input-output equipment such as a keyboard, a mouse, a display, or a printer is connected.

The program P can be recorded on a non-transitory tangible recording medium M capable of being read by the computer C. Examples of such a recording medium M can include a tape, a disk, a card, a semiconductor memory, and a programmable logic circuit. The computer C can obtain the program P via such a recording medium M. The program P can be transmitted via a transmission medium. Examples of such a transmission medium can include a communication network and a broadcast wave. The computer C can also obtain the program P via such a transmission medium.

### [Additional Remark A]

The whole or part of the example embodiments disclosed above can be described as, but not limited to, the following supplementary notes.

### (Supplementary Note A1)

An information processing apparatus, including: a target detecting means for detecting, based on a state of a member of a group, a target member in need of care provided by one or more other members of the group, the state being inferred from an image of the member; and a notifying means for notifying each member of the group of the target member detected.

### (Supplementary Note A2)

The information processing apparatus described in supplementary note A1, further including an provision detecting means for detecting provision, by a member of the group, of care for the target member, the notifying means being configured to end notification regarding the target member on condition that provision of care for the target member is detected.

### (Supplementary Note A3)

The information processing apparatus described in supplementary note A1 or A2, in which the notifying means is configured to, from among icons which correspond to respective members of the group and which are displayed on a display screen that the respective members of the group are allowed to browse, change a display position of an icon of the target member to a position within a predetermined display area set on the display area.

### (Supplementary Note A4)

The information processing apparatus described in any one of supplementary notes A1 to A3, further including a divergence detecting means for detecting a divergence between a result of self-evaluation, performed by the member, of a state of the member and a state of the member inferred from the image, the notifying means being configured to provide notification to the member for which the divergence is detected, the notification urging the member to be careful.

### (Supplementary Note A5)

The information processing apparatus described in any one of supplementary notes A1 to A4, in which a business operator is included in the group as the member, the information processing apparatus further includes a business operator detecting means for detecting a business operator pertaining to care for the target member from among business operators each included in the group as the member, and the notifying means is configured to notify the member who has provided care for the target member, of the business operator detected.

### (Supplementary Note A6)

The information processing apparatus described in any one of supplementary notes A1 to A5, further including a state inferring means for inferring the state of the member from an image captured during a video call made by the member and used for the video call.

### (Supplementary Note A7)

The information processing apparatus described in any one of supplementary notes A1 to A6, further including a priority assigning means for assigning a priority to each of the one or more other members in terms of providing care for the target member, the notifying means being configured to notify, based on the priority, each member of the group.

### (Supplementary Note A8)

The information processing apparatus described in any one of supplementary notes A1 to A7, in which the notifying means is configured to provide notification of a message which is generated with use of a language model trained by machine learning and which describes a state of the target member.

### (Supplementary Note A9)

A mutual watching system, including: the information processing apparatus described in any one of supplementary notes A1 to A7; and a terminal for presenting, based on notification provided by the information processing apparatus, the target member to a member who uses the terminal.

### [Additional Remark B]

### (Supplementary Note B1)

A mutual watching method, including: at least one processor detecting, based on a state of a member of a group, a target member in need of care provided by one or more other members of the group, the state being inferred from an image of the member; and the at least one processor notifying each member of the group of the target member detected.

### (Supplementary Note B2)

The mutual watching method described in supplementary note B1, further including the at least one processor detecting provision, by a member of the group, of care for the target member, in which in the notifying, the at least one processor ends notification regarding the target member on condition that provision of care for the target member is detected.

### (Supplementary Note B3)

The mutual watching method described in supplementary note B1 or B2, in which in the notifying, from among icons which correspond to respective members of the group and which are displayed on a display screen that the respective members of the group are allowed to browse, the at least one processor changes a display position of an icon of the target member to a position within a predetermined display area set on the display area.

### (Supplementary Note B4)

The mutual watching method described in any one of supplementary notes B1 to B3, further including the at least one processor detecting a divergence between a result of self-evaluation, performed by the member, of a state of the member and a state of the member inferred from the image, in which in the notifying, the at least one processor provides notification to the member for which the divergence is detected, the notification urging the member to be careful.

### (Supplementary Note B5)

The mutual watching method described in any one of supplementary notes B1 to B4, in which a business operator is included in the group as the member, the mutual watching method further includes the at least one processor detecting a business operator pertaining to care for the target member from among business operators each included in the group as the member, and in the notifying, the at least one processor notifies the member who has provided care for the target member, of the business operator detected.

### (Supplementary Note B6)

The mutual watching method described in any one of supplementary notes B1 to B5, further including the at least one processor inferring the state of the member from an image captured during a video call made by the member and used for the video call.

### (Supplementary Note B7)

The mutual watching method described in any one of supplementary notes B1 to B6, further including the at least one processor assigning a priority to each of the one or more other members in terms of providing care for the target member, in which in the notifying, the at least one processor notifies, based on the priority, each member of the group.

### (Supplementary Note B8)

The mutual watching method described in any one of supplementary notes B1 to B7, in which in the notifying, the at least one processor provides notification of a message which is generated with use of a language model trained by machine learning and which describes a state of the target member.

### [Additional Remark C]

### (Supplementary Note C1)

A control program for causing a computer to function as an information processing apparatus, the control program causing the computer to function as: a target detecting means for detecting, based on a state of a member of a group, a target member in need of care provided by one or more other members of the group, the state being inferred from an image of the member; and a notifying means for notifying each member of the group of the target member detected.

### (Supplementary Note C2)

The control program described in supplementary note C1, causing the computer to further function as a provision detecting means for detecting provision, by a member of the group, of care for the target member, the notifying means being configured to end notification regarding the target member on condition that provision of care for the target member is detected.

### (Supplementary Note C3)

The control program described in supplementary note C1 or C2, in which the notifying means is configured to, from among icons which correspond to respective members of the group and which are displayed on a display screen that the respective members of the group are allowed to browse, change a display position of an icon of the target member to a position within a predetermined display area set on the display area.

### (Supplementary Note C4)

The control program described in any one of supplementary notes C1 to C3, causing the computer to further function as a divergence detecting means for detecting a divergence between a result of self-evaluation, performed by the member, of a state of the member and a state of the member inferred from the image, the notifying means being configured to provide notification to the member for which the divergence is detected, the notification urging the member to be careful.

### (Supplementary Note C5)

The control program described in any one of supplementary notes C1 to C4, in which a business operator is included in the group as the member, the control program causes the computer to further function as a business operator detecting means for detecting a business operator pertaining to care for the target member from among business operators each included in the group as the member, and the notifying means is configured to notify the member who has provided care for the target member, of the business operator detected.

### (Supplementary Note C6)

The control program described in any one of supplementary notes C1 to C5, causing the computer to further function as a state inferring means for inferring the state of the member from an image captured during a video call made by the member and used for the video call.

### (Supplementary Note C7)

The control program described in any one of supplementary notes C1 to C6, causing the computer to further function as a priority assigning means for assigning a priority to each of the one or more other members in terms of providing care for the target member, the notifying means being configured to notify, based on the priority, each member of the group.

### (Supplementary Note C8)

The control program described in any one of supplementary notes C1 to C7, in which the notifying means is configured to provide notification of a message which is generated with use of a language model trained by machine learning and which describes a state of the target member.

### [Additional Remark D]

### (Supplementary Note D1)

An information processing apparatus, including at least one processor, the at least one processor carrying out: a target detecting process of detecting, based on a state of a member of a group, a target member in need of care provided by one or more other members of the group, the state being inferred from an image of the member; and a notifying process of notifying each member of the group of the target member detected.

### (Supplementary Note D2)

The information processing apparatus described in supplementary note D1, in which the at least one processor further carries out an provision detecting process of detecting provision, by a member of the group, of care for the target member, and in the notifying process, the at least one processor ends notification regarding the target member on condition that provision of care for the target member is detected.

### (Supplementary Note D3)

The information processing apparatus described in supplementary note D1 or D2, in which in the notifying process, from among icons which correspond to respective members of the group and which are displayed on a display screen that the respective members of the group are allowed to browse, the at least one processor changes a display position of an icon of the target member to a position within a predetermined display area set on the display area.

### (Supplementary Note D4)

The information processing apparatus described in any one of supplementary notes D1 to D3, in which the at least one processor further carries out a divergence detecting process of detecting a divergence between a result of self-evaluation, performed by the member, of a state of the member and a state of the member inferred from the image, and in the notifying process, the at least one processor provides notification to the member for which the divergence is detected, the notification urging the member to be careful.

### (Supplementary Note D5)

The information processing apparatus described in any one of supplementary notes D1 to D4, in which a business operator is included in the group as the member, the at least one processor further carries out a business operator detecting process of detecting a business operator pertaining to care for the target member from among business operators each included in the group as the member, and in the notifying process, the at least one processor notifies the member who has provided care for the target member, of the business operator detected.

### (Supplementary Note D6)

The information processing apparatus described in any one of supplementary notes D1 to D5, in which the at least one processor further carries out a state inferring process of inferring the state of the member from an image captured during a video call made by the member and used for the video call.

### (Supplementary Note D7)

The information processing apparatus described in any one of supplementary notes D1 to D6, in which the at least one processor further carries out a priority assigning process of assigning a priority to each of the one or more other members in terms of providing care for the target member, and in the notifying process, the at least one processor notifies, based on the priority, each member of the group.

### (Supplementary Note D8)

The information processing apparatus described in any one of supplementary notes D1 to D7, in which in the notifying process, the at least one processor provides notification of a message which is generated with use of a language model trained by machine learning and which describes a state of the target member.

### [Additional Remark E]

The information processing apparatus may further include a memory. The memory may have stored therein a control program for causing the at least one processor to carry out each of the above processes.

### (Supplementary Note E1)

A computer-readable non-transitory recording medium having recorded thereon a control program for causing a computer to: function as an information processing apparatus, the control program causing the computer to carry out: a target detecting process of detecting, based on a state of a member of a group, a target member in need of care provided by one or more other members of the group, the state being inferred from an image of the member; and a notifying process of notifying each member of the group of the target member detected.

Reference Signs List

1: Information processing apparatus
101: Target detecting section (target detecting means)
102: Notifying section (notifying means)
1A: Information processing apparatus
101A: Target detecting section (target detecting means)
102A: Notifying section (notifying means)
104A: State inferring section (state inferring means)
105A: Priority assigning section (priority assigning means)
106A: Provision detecting section (provision detecting means)
107A: Divergence detecting section (divergence detecting means)
108A: Business operator detecting section
(business operator detecting means)
2: Terminal
3: Mutual watching system

1. An information processing apparatus, comprising
at least one processor, the at least one processor carrying out:
for each member of a group, calculating a value indicating a state of the member based on an image of a face of the member, the state including at least one selected from the group consisting of stress level, degree of concentration, cognitive function, feelings, degree of arousal, and degree of tension;
detecting, as a target member in need of care provided by one or more other members of the group, a member with the value calculated which is equal to or greater than a predetermined threshold; and
sending an instruction to change a display position of an icon of the target member to a position within a predetermined display area set on the display area, from among icons which correspond to respective members of the group and which are displayed on a display screen that the respective members of the group are allowed to browse.

2. The information processing apparatus according to claim 1, wherein
the at least one processor further carries out a provision detecting process of detecting provision, by a member of the group, of care for the target member, and
the at least one processor ends notification regarding the target member on condition that provision of care for the target member is detected.

3. The information processing apparatus according to claim 1, wherein
the at least one processor further carries out a divergence detecting process of detecting a divergence between a result of self-evaluation, performed by the member, of a state of the member and a state of the member inferred from the image, and
the at least one processor provides notification to the member for which the divergence is detected, the notification urging the member to be careful.

4. The information processing apparatus according to claim 1, wherein
a business operator is included in the group as the member,
the at least one processor further carries out a business operator detecting process of detecting a business operator pertaining to care for the target member from among business operators each included in the group as the member, and
the at least one processor notifies the member who has provided care for the target member, of the business operator detected.

5. The information processing apparatus according to claim 1, wherein
the at least one processor further carries out a state inferring process of inferring the state of the member from an image captured during a video call made by the member and used for the video call.

6. The information processing apparatus according to claim 1, wherein
the at least one processor further carries out a priority assigning process of assigning a priority to each of the one or more other members in terms of providing care for the target member, and
in the notifying process, the at least one processor notifies, based on the priority, each member of the group.

7. The information processing apparatus according to claim 1, wherein
the at least one processor provides notification of a message which is generated with use of a language model trained by machine learning and which describes a state of the target member.

8. A mutual watching method, comprising:
at least one processor calculating, for each member of a group, a value indicating a state of the member based on an image of a face of the member, the state including at least one selected from the group consisting of stress level, degree of concentration, cognitive function, feelings, degree of arousal, and degree of tension;
the at least one processor detecting, as a target member in need of care provided by one or more other members of the group, a member with the value calculated which is equal to or greater than a predetermined threshold; and
the at least one processor sending an instruction to change a display position of an icon of the target member to a position within a predetermined display area set on the display area, from among icons which correspond to respective members of the group and which are displayed on a display screen that the respective members of the group are allowed to browse.

9. A computer-readable non-transitory recording medium having recorded thereon a control program for causing a computer to carry out:
for each member of a group, calculating a value indicating a state of the member based on an image of a face of the member, the state including at least one selected from the group consisting of stress level, degree of concentration, cognitive function, feelings, degree of arousal, and degree of tension;
detecting, as a target member in need of care provided by one or more other members of the group, a member with the value calculated which is equal to or greater than a predetermined threshold; and
sending an instruction to change a display position of an icon of the target member to a position within a predetermined display area set on the display area, from among icons which correspond to respective members of the group and which are displayed on a display screen that the respective members of the group are allowed to browse.

10. A mutual watching system, comprising:
the information processing apparatus according to claim 1; and
a terminal for presenting, based on notification provided by the information processing apparatus, the target member to a member who uses the terminal.
